(19) United States
(12) Patent Application Publication
(10) Pub. No.: US 2015/0335367 A1
(43) Pub. Date: Nov. 26, 2015

(54) MEDICAL GRADE COTTON AND EVANS OSTEOTOMY WEDGES

(71) Applicant: NEUTIN ORTHOPEDICS, LLC, Millersville, MD (US)

(72) Inventors: Albert Eugene Austin, MILLERSVILLE, MD (US); Steven Keith Neufeld, Washington, DC (US)

(21) Appl. No.: 14/718,054

(22) Filed: May 20, 2015

Related U.S. Application Data
(60) Provisional application No. 61/997,027, filed on May 20, 2014.

(51) Int. Cl. A61B 17/80; A61F 2/28

(52) U.S. Cl. CPC A61B 17/8095; A61F 2/28

(57) ABSTRACT

A medical grade thermoplastic or polymer material wedge used for various deformity corrections in foot and/or ankle bone surgery of adults and/or children. The medical grade thermoplastic or polymer material wedges can be provided in various predetermined (e.g., prefabricated) sizes for selection therefrom depending on the anatomy and/or desired correction of the deformity when performing an Evans or Cotton corrective osteotomy procedure.

### CROSS-REFERENCE TO RELATED APPLICATIONS

The present application claims the benefit of U.S. Provisional Application No. 61/997,027, filed May 20, 2014, which is hereby incorporated by reference herein in its entirety.

### FIELD

This present disclosure relates generally to medical implants, and, more particularly, to medical grade Cotton and Evans osteotomy wedges.

### BACKGROUND

For many years, orthopedic surgeons and surgical podiatry doctors have been creating ways to surgically correct a patients flatfoot deformity. Flatfoot deformity is an imbalance of both the medial (inside of the foot) and lateral (outside of the foot) column which are the bones on both sides of the foot. One such way to correct this imbalance is to perform an Evans osteotomy, which is an osteotomy of the calcaneas or the heel bone. An osteotomy is a surgical operation whereby a bone is cut to shorten, lengthen, or change its alignment. Evans first introduced the idea of medial and lateral column imbalance, as it applies to talipes equinovarus, in 1961. In this case he described the lateral column as long in comparison to the medial column. Evans later performed a calcaneal lengthening procedure to correct an early post-operative complication in a residual clubfoot patient and reported this in the orthopedic literature in 1975. The Evans osteotomy, as it is called now, is a lateral based opening wedge osteotomy that effectively lengthens the lateral column thus reducing forefoot abduction and transverse plane deformity. It offers triplanar correction of the symptomatic flexible flatfoot by adducting and plantarflexing the forefoot and supinating the subtalar joint.

Another such way to surgically correct a patients flatfoot deformity is the Cotton osteotomy. The Cotton osteotomy is a medial opening wedge osteotomy that is performed in the first cuneiform bone of the foot. Like the Evans, the Cotton osteotomy is a surgically corrective osteotomy to assist in the correction of the flatfoot deformity. Like the Evans, the Cotton osteotomy is an opening wedge osteotomy where the first cuneiform bone is “opened” by the osteotomy cut of the bone to obtain a certain amount of correction. The Cotton osteotomy is a powerful surgical procedure in the treatment of collapsing pes planovalgus with persistent rigid forefoot varus deformity.

Both the Evans and Cotton osteotomy procedures are “opening wedge” osteotomies whereas the bone, by using a surgical saw blade, is surgically cut open with an osteotomy to achieve correction, as described above. When you open the bones, the surgeon needs the bone to stay in the open position to maintain and hold the corrective procedure they have performed.

Various methods of maintaining the corrections of both Cotton and Evans osteotomies have been studied; from metal plates to bone molds that are inserted into the osteotomy to hold the correction. The bone graft molds or wedges have varied from autogenous bone (bone harvested from the patient's own body, often from the iliac crest), allograft (cadaveric bone usually obtained from a bone bank), or synthetic (often made of hydroxyapatite or other naturally occurring and biocompatible substances) with similar mechanical properties to bone. Most bone grafts are expected to be reabsorbed and replaced as the natural bone heals over a few months' time. Recently, titanium and porous metal alloy wedges have been used to replicate the opening space of the Cotton or Evans osteotomy and they inserted into the osteotomy opening similar to a bone graft mold or wedges.

### SUMMARY

In one or more embodiments, an osteotomy implant for surgical foot and/or ankle osteotomy bone corrections comprises a first end, a second end opposite the first end, an opening at the second end, and a threaded hole at the first end. The first end can be thicker than the second end in a side view. The opening can extend toward the first end in a plan view. The osteotomy implant can be composed of one or more medical-grade materials, the one or more medical-grade materials being radiolucent and/or osteoconductive. The osteotomy implant can be adapted to hold a bone correction achieved by an osteotomy cut. The threaded hole can be threaded for attachment of an insertion tool.

In one or more embodiments, a method comprises making an osteotomy cut of a bone to achieve a correction. The method can also include selecting a prefabricated osteotomy wedge implant based on one or more characteristics of the cut, and inserting the selected prefabricated osteotomy wedge implant into the cut to maintain the achieved bone correction. The selected prefabricated osteotomy wedge implant comprises a first end, a second end opposite the first end, and an opening at the second end. The opening can extend toward the first end in plan view. The selected prefabricated osteotomy wedge implant can be composed of one or more medical-grade materials, the one or more medical-grade materials being radiolucent and/or osteoconductive. The selected prefabricated osteotomy wedge implant can be adapted to maintain the bone correction achieved by the osteotomy cut.

In one or more embodiments, a method comprises making an osteotomy cut of a bone of a patient to achieve a correction. The method can also comprise selecting a trial wedge based on a characteristic of the cut, the patient, and/or a desired correction. The method can also comprise inserting the trial wedge into the cut. The method can also comprise determining whether an actual correction achieved with the trial wedge is acceptable. The method can also comprise, selecting, when the actual correction achieved with the trial wedge is acceptable, a prefabricated osteotomy wedge implant based on a characteristic of the accepted trial wedge. The method can also comprise inserting the selected prefabricated osteotomy wedge implant into the cut to maintain the achieved bone correction. The selected prefabricated osteotomy wedge implant comprises a first end, a second end opposite the first end, and an opening at the second end. The opening can extend toward the first end in plan view. The selected prefabricated osteotomy wedge implant can be composed of one or more medical-grade materials, the one or more medical-grade materials being radiolucent and/or osteoconductive. The selected prefabricated osteotomy wedge implant can be adapted to maintain the bone correction achieved by the osteotomy cut.

In one or more embodiments, an osteotomy wedge kit for surgical foot and/or ankle osteotomy bone corrections comprises a plurality of osteotomy implants and a plurality of trial wedges. The plurality of osteotomy implants can be of two or more types/sizes. Each of the trial wedges can be configured to represent a respective one of the types/sizes of the implants. Each osteotomy implant can comprise a first end, a second end opposite the first end, and an opening at the second end. The opening can extend toward the first end in a plan view. Each osteotomy implant can be composed of one or more medical-grade materials, the one or more medical-grade materials being radiolucent and/or osteoconductive. Each osteotomy implant can be adapted to hold a bone correction achieved by an osteotomy cut.

Objects and advantages of embodiments of the disclosed subject matter will become apparent from the following description when considered in conjunction with the accompanying drawings.

### BRIEF DESCRIPTION OF THE DRAWINGS

Embodiments will hereinafter be described with reference to the accompanying drawings, which have not necessarily been drawn to scale. Where applicable, some features may not be illustrated to assist in the illustration and description of underlying features. Throughout the figures, like reference numerals denote like elements. As used herein, various embodiments can mean one, some, or all embodiments.

FIG. 1 is a top plan view of a medical grade thermoplastic or polymer osteotomy wedge, according to one or more embodiments of the disclosed subject matter.

FIG. 2 is a front side view of the medical grade thermoplastic or polymer osteotomy wedge shown in FIG. 1, according to one or more embodiments of the disclosed subject matter.

FIG. 3 is a back side view of the medical grade thermoplastic or polymer osteotomy wedge shown in FIG. 1, according to one or more embodiments of the disclosed subject matter.

FIG. 4 is a left/angled side view of the medical grade thermoplastic or polymer osteotomy wedge shown in FIG. 1, according to one or more embodiments of the disclosed subject matter.

FIG. 5 is a side view of the medical grade thermoplastic or polymer osteotomy wedge shown in FIG. 1, according to one or more embodiments of the disclosed subject matter.

FIG. 6 illustrates a process flow for a method of using a prefabricated osteotomy wedge implant, according to one or more embodiments of the disclosed subject matter.

FIG. 7 is a top plan view of a medical grade osteotomy wedge implant, according to one or more embodiments of the disclosed subject matter.

FIG. 8 is a back side view of the medical grade osteotomy wedge implant shown in FIG. 7, according to one or more embodiments of the disclosed subject matter.

FIG. 9 is a top/angled side view of the medical grade osteotomy wedge implant shown in FIG. 7, according to one or more embodiments of the disclosed subject matter.

FIG. 10 is a side view of the medical grade osteotomy wedge implant shown in FIG. 7, according to one or more embodiments of the disclosed subject matter.

FIG. 11 is a top plan view of a medical grade osteotomy wedge implant, according to one or more embodiments of the disclosed subject matter.

FIG. 12 is a back side view of the medical grade osteotomy wedge implant shown in FIG. 11, according to one or more embodiments of the disclosed subject matter.

FIG. 13 is a top/angled side view of the medical grade osteotomy wedge implant shown in FIG. 11, according to one or more embodiments of the disclosed subject matter.

FIG. 14 is a side view of the medical grade osteotomy wedge implant shown in FIG. 11, according to one or more embodiments of the disclosed subject matter.

### DETAILED DESCRIPTION

The present inventors have recognized that the prior art does not disclose a medical grade thermoplastic or polymer osteotomy wedge used for various deformity corrections in foot and ankle bone surgery of adults and children. By the present application there is provided either a pre-determined or fabricated shape wedge made out of a medical grade thermoplastic or polymer material used for various deformity corrections in foot and ankle bone surgery of adults or children. Some embodiments include titanium alloy or other allow (e.g. porous metal alloy) osteotomy wedges. In some embodiments, one or more surfaces of the osteotomy wedge are coated with an osteoconductive coating such as, for example, a hydroxyapatite (HAp) coating.

The present disclosure overcomes deficiencies of possible complications using bone graft wedges and some embodiments provide radiolucency compared to metal wedges.

In one or more embodiments, a medical grade thermoplastic or polymer osteotomy wedge used for various flatfoot deformity corrections in bone surgery of adults and children is made from medical grade thermoplastic or polymer material, some of which have shown to be osteoconductive and radiolucent. The medical grade thermoplastic or polymer osteotomy wedge used for various flatfoot deformity corrections in bone surgery of adults and children can pre-determined or fabricated and may come in a variety of sizes depending on the required deformity correction. In one or more embodiments, at least one surface of the osteotomy wedge is coated with an osteoconductive material or compound such as, for example, hydroxyapatite (HAp).

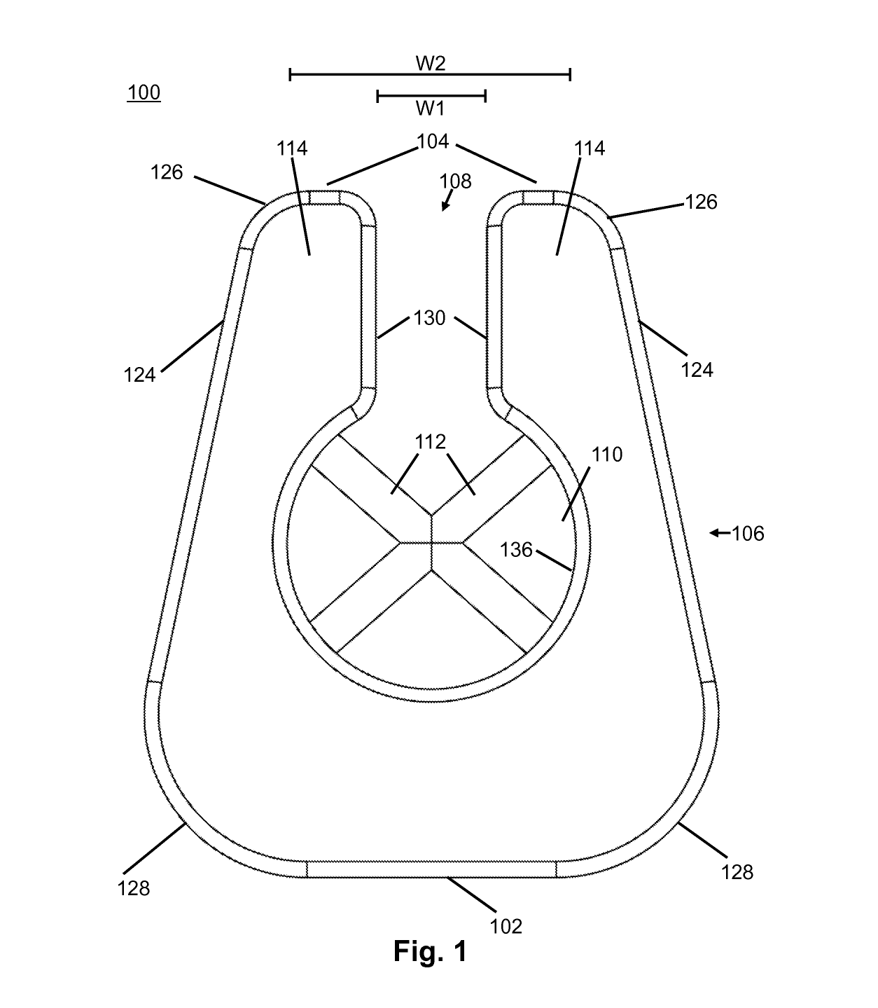

In some embodiments, medical grade materials/polymers are used to replicate bone as an implant. There are various medically accepted materials/polymers including but not limited to polyetheretherketone (PEEK), polyehterketoneketone (PEKK), Carbon Fiber-PEKK combination, and other polymer composite material that have passed the review of the U.S. Food and Drug Administration (FDA) allowing them to be used as a medical implant. Some of these polymer composites have shown to be osteoconductive. In some embodiments, one or more surfaces are coated with an osteoconductive material or compound such as, for example, hydroxyapatite (HAp). Osteoconductivity is the process by which bone grows on a surface (e.g., new bone growth that is perpetuated by the native bone). Some such medical grade polymers have met the stringent manufacturing guidelines ISO 10993 biocornpatibility testing along with other accepted manufacturing and biocornpatibility guidelines. Medical grade polymers have the advantage of being able to be molded into any shape or design desired, such as, for example, those shown in FIGS. 1-5 and 7-14 and discussed below.

For purposes of promoting an understanding of the principles of the present disclosure, reference will now be made to the examples illustrated in the drawings and described in the following written specification. It is understood that no limitation to the scope of the present disclosure is thereby intended. It is further understood that the present disclosure includes any alteration and modifications to the illustrated examples and includes further applications of the principles disclosed herein as would normally occur to one skilled in the art to which this disclosure pertains.

FIGS. 1-5 and 7-14 depict embodiments of medical grade osteotomy wedges used for various flatfoot deformity corrections in foot and/or ankle bone surgery of adults and/or children.

FIG. 1 is a top plan view of a medical grade thermoplastic or polymer osteotomy wedge 100, according to one or more embodiments of the disclosed subject matter. Osteotomy wedge 100 can be used for various flatfoot deformity corrections in foot and/or ankle bone surgery of adults and children. Osteotomy wedge 100 includes a first end 102, a second end 104, a middle portion 106, lattice 112, members 114, side surfaces 124, first end corner surfaces 128, second end corner surfaces 126, inner surfaces 130, and curved surface 136. An opening 108 is formed at the second end 104 and separates the second end 104 into two portions 114. End portions 114 are spaced apart in plan view. The opening 108 extends toward the first end 102 in plan view and increases in width in plan view at a middle portion 106 to form a larger opening 110 that is semicircular (or substantially semicircular) in plan view. Lattice 112 is coupled to curved surface 136 which forms (or surrounds) the semicircular opening 110. The width W2 (or diameter) of the semicircular opening 110 is larger than the width W1 of the opening 108.

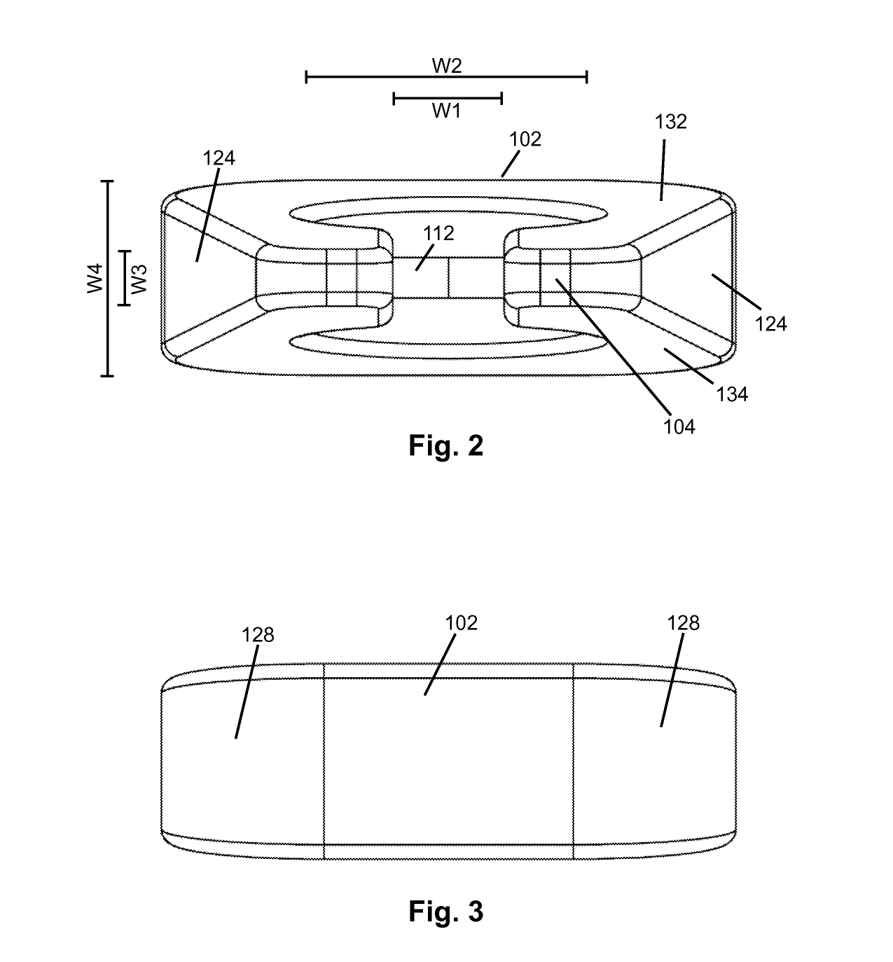

Osteotomy wedge 102 is tapered in plan view from the first end 102 to the second end 104 (i.e., side surfaces 124 are angled inward from the first end 102 to the second end 104). The osteotomy wedge 100 is thicker at the first end 102 than the second end 104, as illustrated, for example, in FIGS. 2, 4, and 5.

Corner surfaces 128 and 126 are curved and corner surfaces 128 have a larger curvature than corner surfaces 126. In some embodiments, corner surfaces 128 have a curvature 2.25 times larger than corner surfaces 126. In some embodiments, corner surfaces 124 can have a curvature twice as large as the curvature of the curved surfaces between the second end 104 and the inner surfaces 130. In some embodiments, corner surfaces 128 can have a curvature 4.5 times as large as the curvature of the curved surfaces between the second end 104 and the inner surfaces 130.

In some embodiments, the center of semicircular opening 110 can be at the center or substantially at the center of osteotomy wedge 100 in plan view. Although shown in FIG. 1 as semicircular in plan view, opening 110 can be formed in any shape in plan view, such as, for example, a square, rectangle, oval, or any other shape.

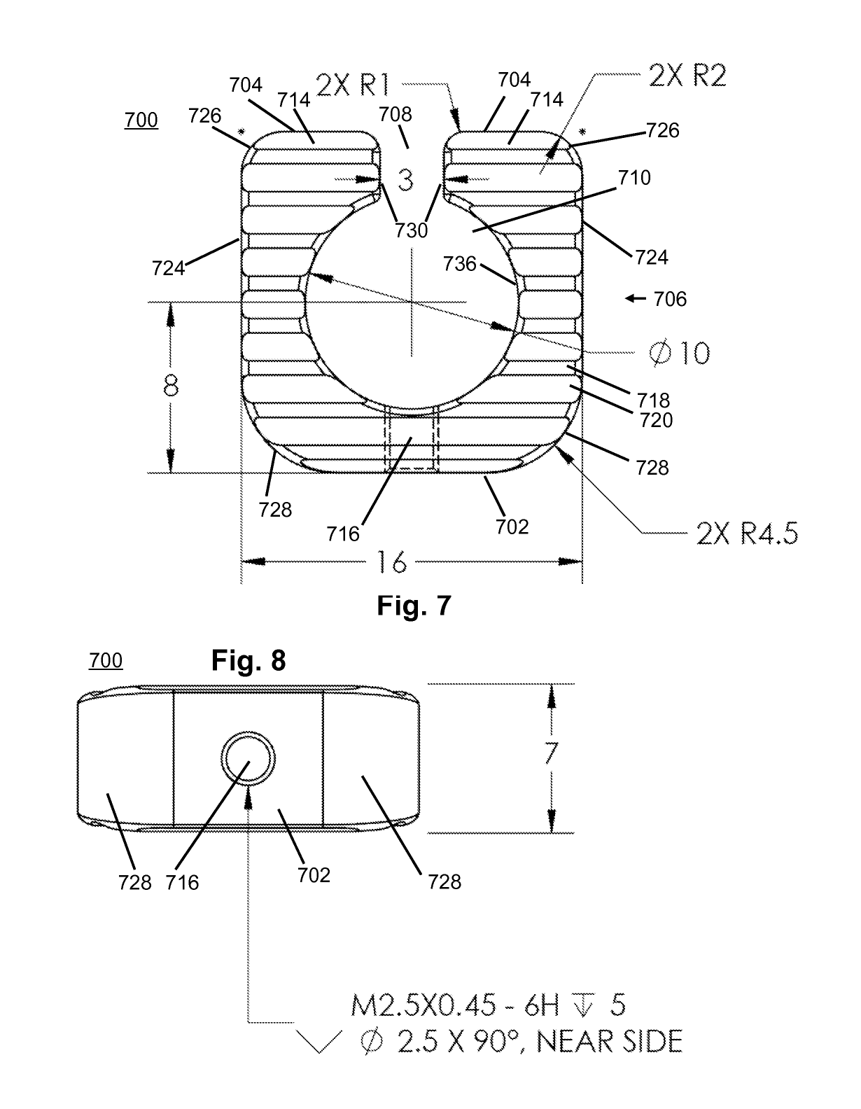

Although portions of lattice 112 are shown in FIGS. 1 and 4 and described herein as cylindrical, such portions of lattice 112 can be formed in other cross sectional shapes such as, for example, square, rectangular, or any other shape. In some embodiments, wedge 100 does not include lattice 112, as shown, for example, by surfaces 724 shown in FIGS. 7-14.

In some embodiments, the side surfaces 124 are not angled inward from the first end 102 to the second end 104. For example, in some such embodiments, side surfaces 124 are parallel or substantially parallel, as shown, for example, in FIGS. 7-14.

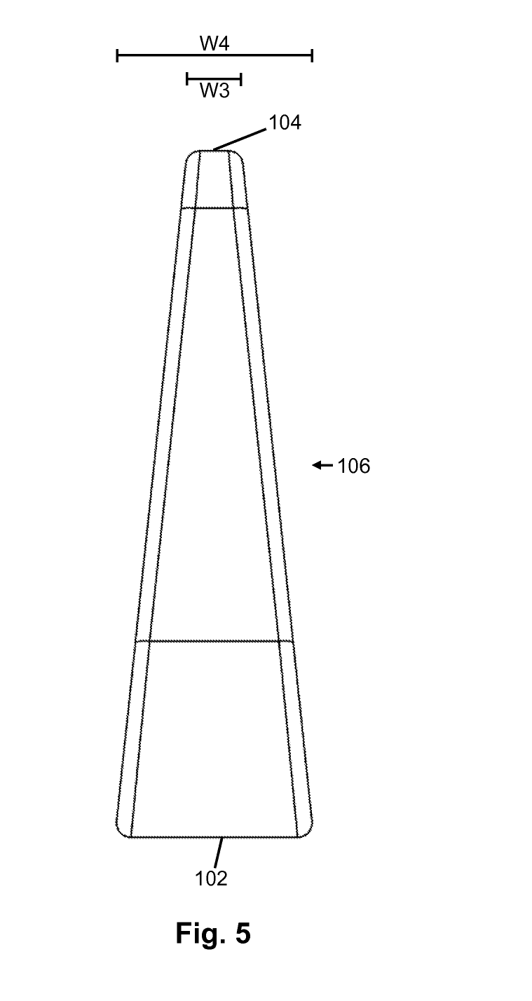

FIG. 2 is a front side view of the medical grade thermoplastic or polymer osteotomy wedge 100 shown in FIG. 1, according to one or more embodiments of the disclosed subject matter; and FIG. 5 is a side view of the medical grade thermoplastic or polymer osteotomy wedge 100 shown in FIG. 1, according to one or more embodiments of the disclosed subject matter. FIGS. 2 and 5 illustrate that osteotomy wedge 100 has a thickness W4 at the first end 102 and a smaller thickness W3 at the second end 104. As shown in FIG. 2, osteotomy wedge 100 includes planar surfaces 132 and 134 which extend in respective planes from the first end 102 to the second end 104.

In some embodiments, planar surfaces 132 and 134 can include features 718/720 shown in FIGS. 7-14 and describe below to improve bone ingrowth and/or provide anti-migration features when wedge 100 is implanted. In some embodiments, surfaces 132 and 134 can be coated with an osteoconductive coating such as, for example, a hydroxyapatite (HAp) coating.

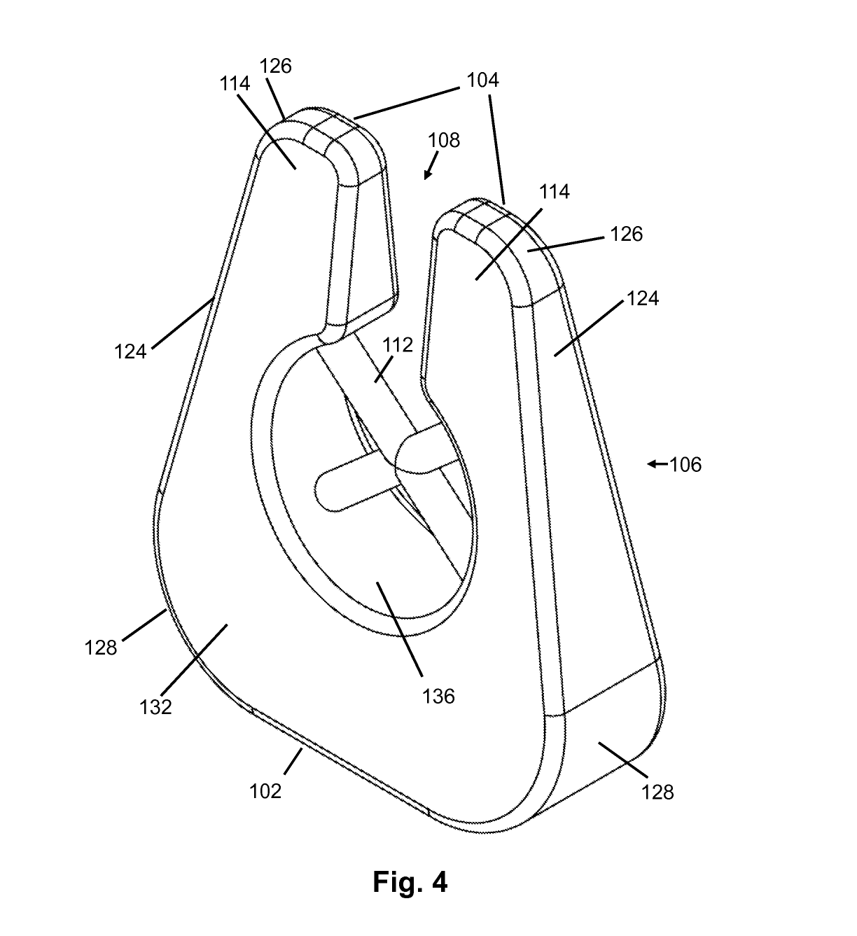

FIG. 3 is a back side view of the medical grade thermoplastic or polymer osteotomy wedge 100 shown in FIG. 1, according to one or more embodiments of the disclosed subject matter; and FIG. 4 is a left/angled side view of the medical grade thermoplastic or polymer osteotomy wedge 100 shown in FIG. 1, according to one or more embodiments of the disclosed subject matter. FIGS. 3 and 4 provide additional views of osteotomy wedge 100.

Although not shown in FIG. 3, in some embodiments, osteotomy wedge 100 includes a tool attachment site such as, for example, threaded hole 716 of osteotomy wedges 700 and 1100 shown in FIGS. 7-14 and described below for attachment of an insertion tool to assist a user when inserting the wedge into an osteotomy cut.

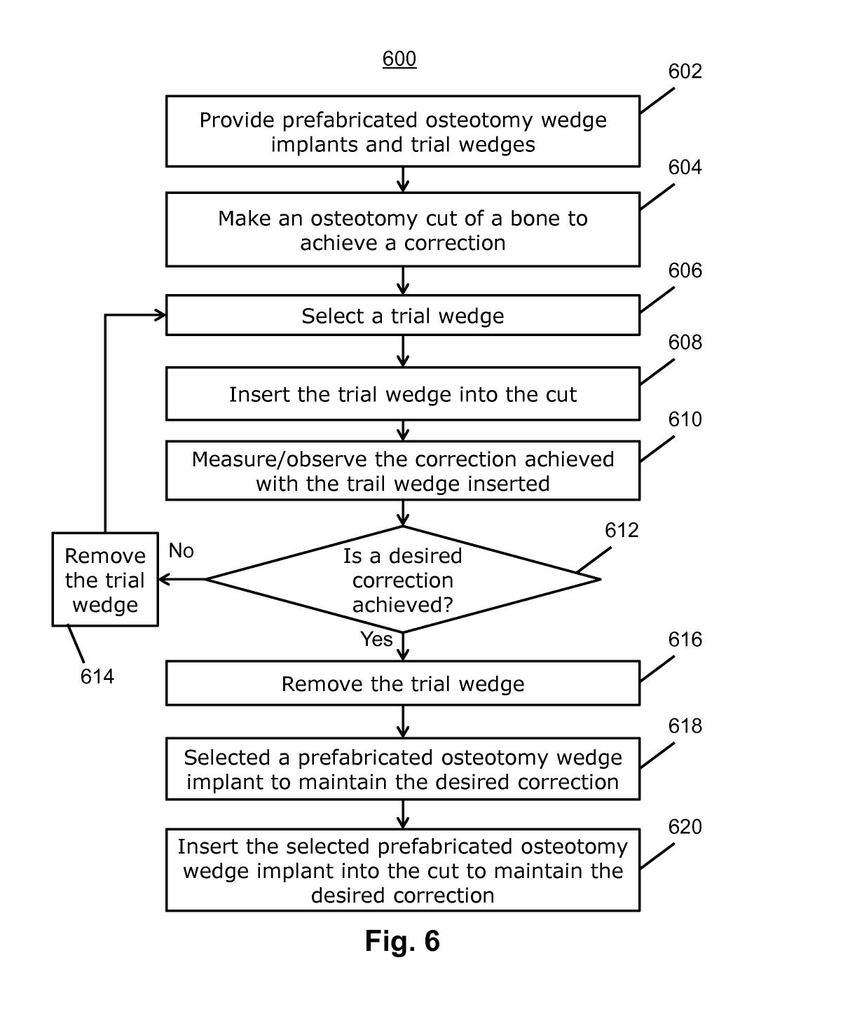

In one or more embodiments, methods for performing an osteotomy (e.g., an Evans or Cotton osteotomy) employ any of the disclosed osteotomy implants or combinations thereof. For example, FIG. 6 illustrates a process flow for a method 600 of using a prefabricated osteotomy wedge implant, according to one or more embodiments of the disclosed subject matter.

At 602, prefabricated osteotomy wedge implants and trial wedges are provided. The implants can be of different sizes, types, and/or configurations. For example, the implants can include various sizes including one or more adult size implants and/or one or more child size implants. The implants can also be of different embodiments of the osteotomy wedge implants disclosed herein including, for example, the implants 100, 700, and 1100 shown in

FIGS. 1-5 and 7-14 and discussed herein. The various sizes can also depend on the amount of correction desired to be achieved. Various types/sizes/configurations can be included for different types of osteotomy procedures to be performed (e.g., Cotton and/or Evans osteotomy procedures). For each of the different types/sizes/configurations of implants provided, a respective trial wedge can be provided to determine the appropriate type/size/configuration of implant to use to achieve a desired correction, as discussed below.

At 604, an osteotomy cut is made of a bone to achieve a desired bone correction. For example, the osteotomy cut can be either a Cotton osteotomy or an Evans osteotomy. The desired bone correction can include, for example, a correction of a flatfoot deformity.

At 606, a trial wedge is selected. The trial wedge can be selected based on one or more characteristics of the cut, patient, and/or desired correction. The trial wedge can be selected from those provided at 602.

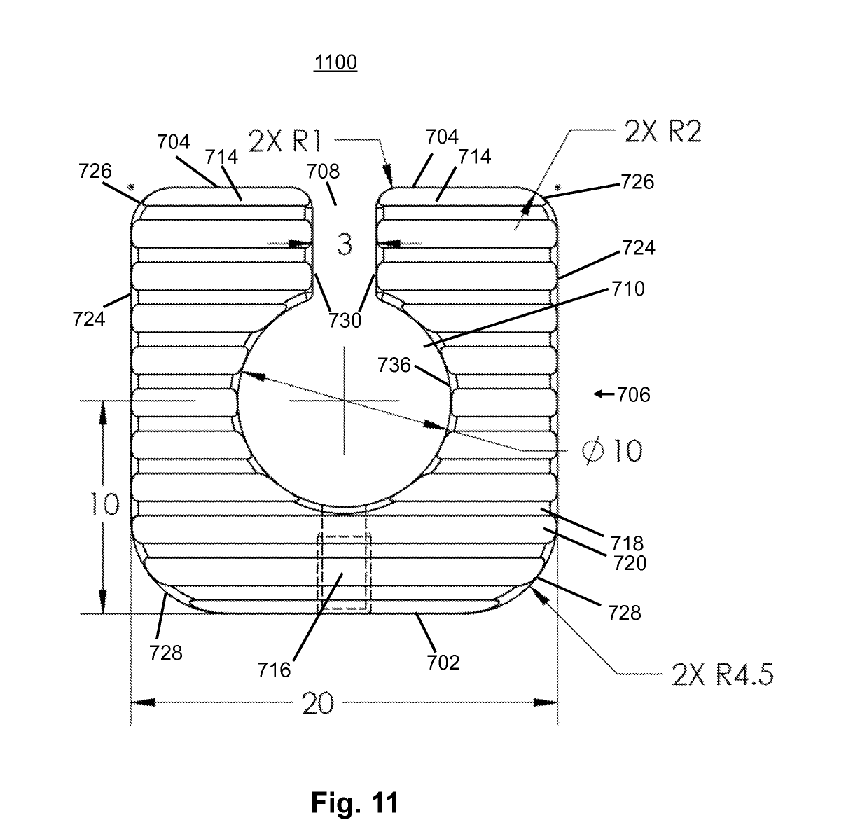

At 608, the trial wedge is inserted into the cut. The trial wedge can include a threaded hole similar to hole 716 of implants 700 and 1100 shown in FIGS. 7 and 11, respectively, and discussed below to accept an insertion tool to assist the user in inserting and removing the trial wedge.

At 610, the correction achieved with the trial wedge inserted is measured and/or observed. For example, the location of the cut (e.g., the foot and/or ankle) can be x-rayed to evaluate the correction achieved.

At 612, it is determined whether a desired correction is achieved. If not, at 614, the trial wedge is removed and another trial wedge is selected at 606.

If it is determined, at 612, that a desired correction is achieved, then the trial wedge is removed at 616.

At 618, a prefabricated osteotomy wedge implant is selected to maintain the desired correction. A prefabricated osteotomy wedge implant can be selected from those provided at 602 based on the selected trial wedge with which a desired correction was achieved. The selected implant can be the type/size/configuration of implant to which the selected trial wedge corresponds.

At 620, the selected prefabricated osteotomy wedge implant is inserted into the cut to maintain the bone correction achieved.

FIG. 7 is a top plan view of a medical grade osteotomy wedge implant 700, according to one or more embodiments of the disclosed subject matter. Osteotomy wedge 700 can be used for various flatfoot deformity corrections in foot and/or ankle bone surgery of adults and children. Osteotomy wedge 700 includes a first end 702, a second end 704, a middle portion 706, end portions 714, hole 716, raised surfaces 718, grooves 720, side surfaces 724, first end corner surfaces 728, second end corner surfaces 726, inner surfaces 730, and curved surface 736. An opening 708 is formed at the second end 704 and separates the second end 704 into two portions 714. End portions 714 are spaced apart in plan view. The opening 708 extends toward the first end 702 in plan view and increases in width in plan view at the middle portion 706 to form a larger opening 710 that is semicircular (or substantially semicircular) in plan view. Hole 716 provides an attachment site for an insertion tool. In some embodiments, hole 716 is threaded to allow a threaded insertion tool to be securely attached to wedge 700 for insertion/removal of the wedge 700 by a user. Although not shown, in some embodiments, wedge 700 includes a lattice coupled to curved surface 736 which forms (or surrounds) the semicircular opening 710 (e.g., lattice 112 of wedge 100 shown in FIGS. 1-5).

Side surfaces 724 are parallel or substantially parallel to each other. Although not shown, in some embodiments, osteotomy wedge 702 is tapered in plan view from the first end 702 to the second end 704 (i.e., side surfaces 724 are angled inward from the first end 702 to the second end 704, as shown, for example, by side surfaces 124 in FIG. 1). The osteotomy wedge 700 is thicker at the first end 702 than the second end 704, as illustrated, for example, in FIGS. 8-10.

Corner surfaces 728 and 726 are curved and corner surfaces 728 have a larger curvature than corner surfaces 726. In some embodiments, corner surfaces 728 have a curvature 2.25 times larger than corner surfaces 726. In some embodiments, corner surfaces 724 can have a curvature twice as large as the curvature of the curved surfaces between the second end 704 and the inner surfaces 730. In some embodiments, corner surfaces 728 can have a curvature 4.5 times as large as the curvature of the curved surfaces between the second end 704 and the inner surfaces 730.

The center of semicircular opening 710 is at the center or substantially at the center of osteotomy wedge 700 in plan view. Although shown in FIG. 7 as semicircular in plan view, opening 710 can be formed in any shape in plan view, such as, for example, a square, rectangle, oval, or any other shape.

Surfaces 718 and/or 720 are configured to improve bone ingrowth and/or provide anti-migration features when implanted. In some embodiments, surfaces 718 and/or 720 can be coated with an osteoconductive coating such as, for example, a hydroxyapatite (HAp) coating.

In some embodiments, osteotomy wedge 700 has dimensions of 16 mm×16 mm in plan view, a thickness at first end 702 of 7 mm, semicircle opening 710 has a diameter of 10 mm, opening 108 has a width of 3 mm, and hole 716 has a diameter of 2.5 mm. In some such embodiments, osteotomy wedge 700 has a thickness at the second end 704 of 2.503 mm or approximately 2.503 mm.

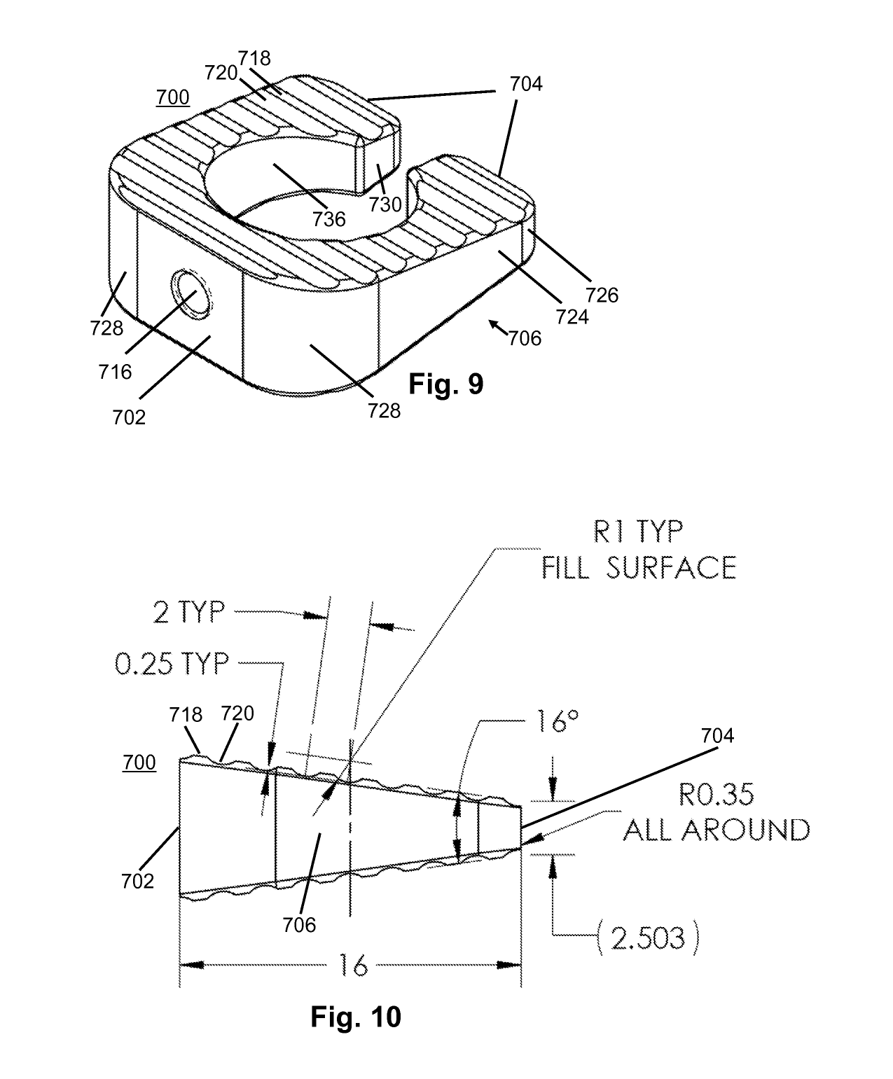

FIG. 8 is a back side view of the medical grade osteotomy wedge implant 700 shown in FIG. 7, according to one or more embodiments of the disclosed subject matter; and FIG. 9 is a top/angled side view of the medical grade osteotomy wedge implant 700 shown in FIG. 7, according to one or more embodiments of the disclosed subject matter. FIGS. 8 and 9 provide additional views of osteotomy wedge implant 700.

FIG. 10 is a side view of the medical grade osteotomy wedge implant 700 shown in FIG. 7, according to one or more embodiments of the disclosed subject matter. In some embodiments, the distance between the centers of adjacent grooves 720 is 2 mm or approximately 2 mm. In some embodiments, raised surfaces 718 extend 0.25 mm above a plane formed along the bottoms of the grooves 720.

FIG. 11 is a top plan view of a medical grade osteotomy wedge implant 1100, according to one or more embodiments of the disclosed subject matter. Osteotomy wedge 1100 can be used for various flatfoot deformity corrections in foot and/or ankle bone surgery of adults and children. Osteotomy wedge 1100 includes a first end 702, a second end 704, a middle portion 706, end portions 714, hole 716, raised surfaces 718, grooves 720, side surfaces 724, first end corner surfaces 728, second end corner surfaces 726, inner surfaces 730, and curved surface 736. An opening 708 is formed at the second end 704 of wedge 1100 and separates the second end 704 into two portions 714. End portions 714 of wedge 1100 are spaced apart in plan view. The opening 708 extends toward the first end 702 of wedge 1100 in plan view and increases in width in plan view at the middle portion 706 to form a larger opening 710 that is semicircular (or substantially semicircular) in plan view. Hole 716 provides an attachment site for an insertion tool. In some embodiments, hole 716 is threaded to allow a threaded insertion tool to be securely attached to wedge 1100 for insertion/removal of the wedge 1100 by a user. Although not shown, in some embodiments, wedge 1100 includes a lattice coupled to curved surface 736 which forms (or surrounds) the semicircular opening 710 (e.g., lattice 112 of wedge 100 shown in FIGS. 1-5).

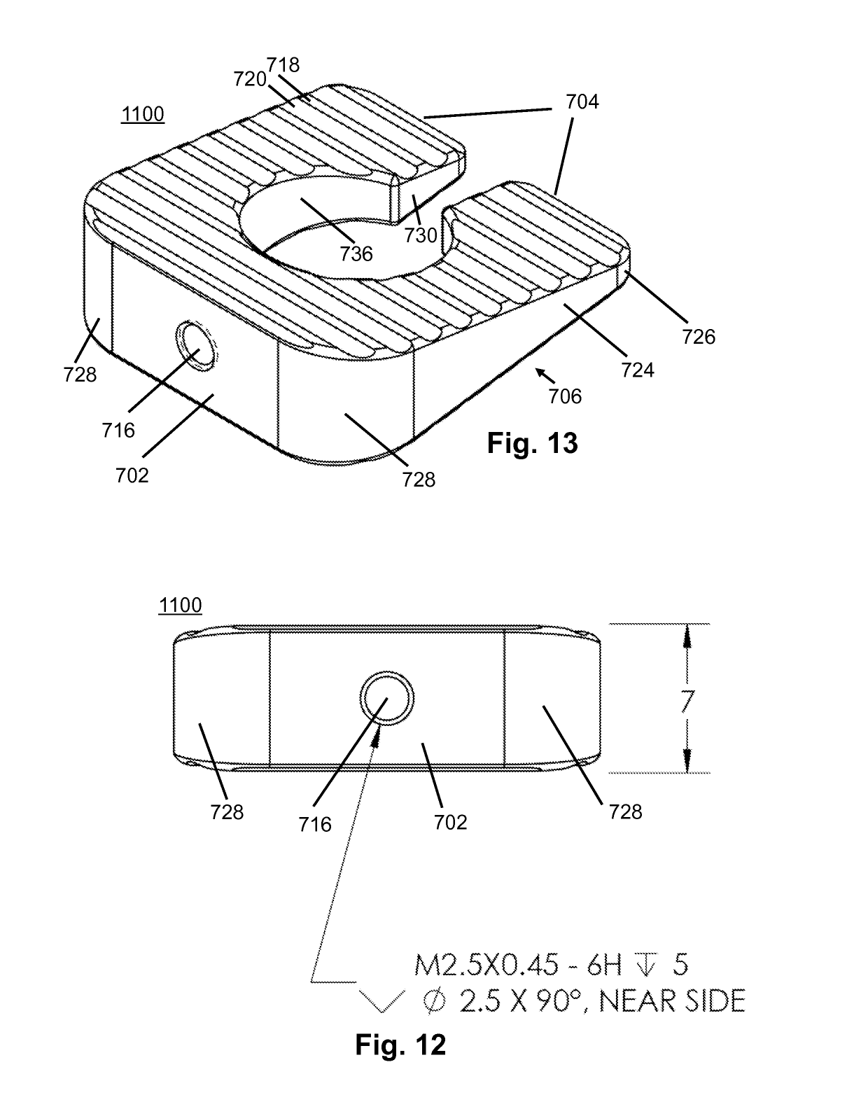

Side surfaces 724 of implant 1100 are parallel or substantially parallel to each other. Although not shown, in some embodiments, osteotomy wedge 1100 is tapered in plan view from the first end 702 to the second end 704 (i.e., side surfaces 724 are angled inward from the first end 702 to the second end 704, as shown, for example, by side surfaces 124 in FIG. 1). The osteotomy wedge 1100 is thicker at the first end 702 than the second end 704, as illustrated, for example, in FIGS. 12-14.

Corner surfaces 728 and 726 of implant 1100 are curved and corner surfaces 728 have a larger curvature than corner surfaces 726. In some embodiments, corner surfaces 728 of implant 1100 have a curvature 2.25 times larger than corner surfaces 726. In some embodiments, corner surfaces 724 of implant 1100 have a curvature twice as large as the curvature of the curved surfaces between the second end 704 and the inner surfaces 730. In some embodiments, corner surfaces 728 of implant 1100 have a curvature 4.5 times as large as the curvature of the curved surfaces between the second end 704 and the inner surfaces 730.

The center of semicircular opening 710 is at the center or substantially at the center of osteotomy wedge 1100 in plan view. Although shown in FIG. 11 as semicircular in plan view, opening 710 can be formed in any shape in plan view, such as, for example, a square, rectangle, oval, or any other shape.

Surfaces 718 and/or 720 are of implant 1100 are configured to improve bone ingrowth and/or provide anti-migration features when implanted. In some embodiments, surfaces 718 and/or 720 of implant 1100 can be coated with an osteoconductive coating such as, for example, a hydroxyapatite (HAp) coating.

In some embodiments, osteotomy wedge 1100 has dimensions of 20 mm×20 mm in plan view, a thickness at first end 702 of 7 mm, semicircle opening 710 has a diameter of 10 mm, opening 108 has a width of 3 mm, and hole 716 has a diameter of 2.5 mm. In some such embodiments, osteotomy wedge 1100 has a thickness at the second end 704 of 1.378 mm or approximately 1.378 mm.

FIG. 12 is a back side view of the medical grade osteotomy wedge implant 1100 shown in FIG. 11, according to one or more embodiments of the disclosed subject matter; and FIG. 13 is a top/angled side view of the medical grade osteotomy wedge implant 1100 shown in FIG. 11, according to one or more embodiments of the disclosed subject matter. FIGS. 12 and 13 provide additional views of osteotomy wedge implant 1100.

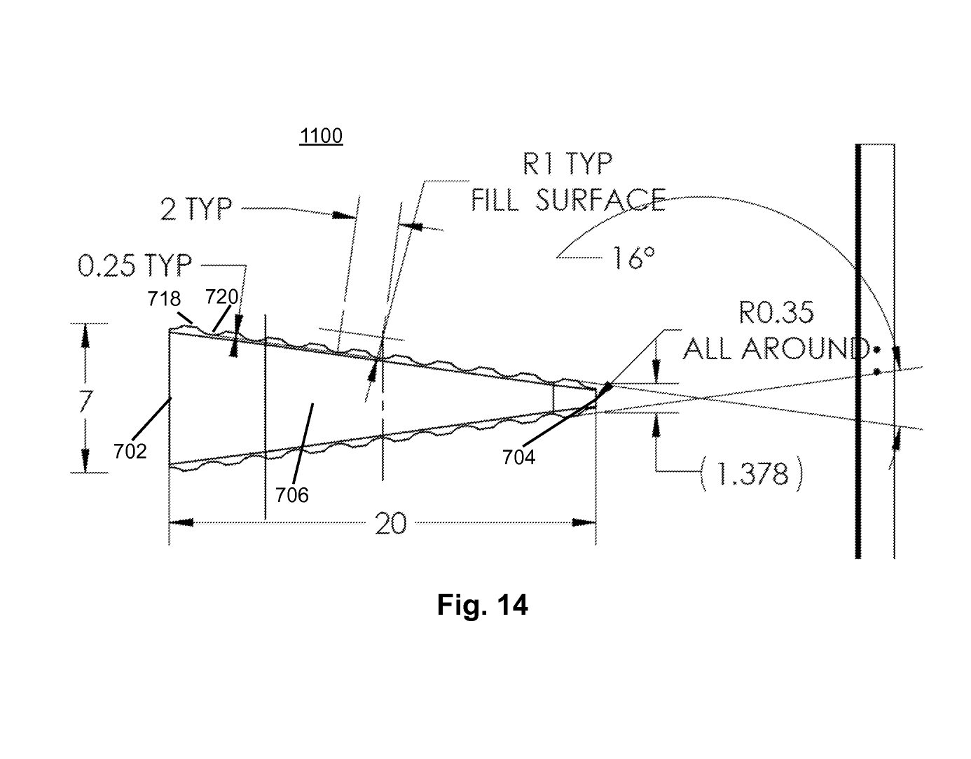

FIG. 14 is a side view of the medical grade osteotomy wedge implant 1100 shown in FIG. 7, according to one or more embodiments of the disclosed subject matter. In some embodiments, the distance between the centers of adjacent ones of grooves 720 of implant 1100 is 2 mm or approximately 2 mm. In some embodiments, raised surfaces 718 of implant 1100 extend 0.25 mm above a plane formed along the low points of grooves 720.

Some embodiments provide the user with a medical grade thermoplastic or polymer osteotomy wedge for application and insertion into an adult or child of a flatfoot correction wedge to stabilize and maintain a Cotton or Evans wedge osteotomy procedure to correct a flatfoot deformity.

Although some embodiments herein have been described with respect to osteotomy wedges/implants for use with a human patient, embodiments of the disclosed subject matter are not limited thereto. Rather, embodiments can include osteotomy wedges/implants for use with an animal, for example.

In some embodiments, the medical grade thermoplastic or polymer osteotomy wedge can be provided in various sizes depending, for example, on the amount of correction required to correct the flatfoot deformity.

Although some embodiments herein have been described with respect to thermoplastic or polymer osteotomy wedges/implants, embodiments of the disclosed subject matter are not limited thereto. Rather, embodiments can include titanium alloy or other alloy (e.g., porous metal alloy) osteotomy wedges/implants.

In this application, unless specifically stated otherwise, the use of the singular includes the plural and the use of “or” means “and/or.” Furthermore, use of the terms “including” or “having,” as well as other forms, such as “includes,” “included,” “has,” or “had” is not limiting. Any range described herein will be understood to include the endpoints and all values between the endpoints.

Features of the disclosed embodiments may be combined, rearranged, omitted, etc., within the scope of the invention to produce additional embodiments. Furthermore, certain features may sometimes be used to advantage without a corresponding use of other features.

It is, thus, apparent that there is provided, in accordance with the present disclosure, medical grade Cotton and Evans osteotomy wedges. Many alternatives, modifications, and variations are enabled by the present disclosure. While specific embodiments have been shown and described in detail to illustrate the application of the principles of the invention, it will be understood that the invention may be embodied otherwise without departing from such principles. Accordingly, Applicant intends to embrace all such alternatives, modifications, equivalents, and variations that are within the spirit and scope of the present invention.

1. An osteotomy implant for surgical foot and/or ankle osteotomy bone corrections, the osteotomy implant comprising:
a first end;
a second end opposite the first end, the first end being thicker than the second end in a side view;
a threaded hole at the first end, the threaded hole being threaded for attachment of an insertion tool;
an opening at the second end, the opening extending toward the first end in a plan view;
the osteotomy implant being composed of one or more medical-grade materials, the one or more medical-grade materials being radiolucent and/or osteoconductive; and
the osteotomy implant being adapted to hold a bone correction achieved by an osteotomy cut.

2. The osteotomy implant of claim 1,
wherein the one or more medical-grade materials are osteoconductive; and
wherein the osteotomy implant comprises a surface adapted to promote bone growth when the osteotomy implant is implanted to hold the bone correction achieved by the osteotomy cut.

3. The osteotomy implant of claim 2, wherein the opening has a first width in the plan view at the second end and a second width in the plan view at a middle portion of the osteotomy implant, the second width being wider than the first width.

4. The osteotomy implant of claim 3, wherein a portion of the opening is semi-circular in the plan view at the middle portion of the osteotomy implant, the second width being a diameter of the semicircular portion of the opening.

5. The osteotomy implant of claim 4, further comprising a surface that is semicircular in the plan view, the semicircular surface forming at least a portion of the semicircular portion of the opening.

6. The osteotomy implant of claim 5, further comprising a member coupled to the semicircular surface and extending across the semi-circular opening in the plan view.

7. The osteotomy implant of claim 1, wherein the one or more medical-grade materials comprises polyetheretherketone (PEEK).

8. The osteotomy implant of claim 1, wherein the one or more medical-grade materials comprises polyetherketoneketone (PEKK).

9. The osteotomy implant of claim 1, wherein the one or more medical-grade materials comprises carbon fiber and polyetherketoneketone (PEKK).

10. The osteotomy implant of claim 1, wherein the osteotomy cut is a Cotton osteotomy.

11. The osteotomy implant of claim 1, wherein the osteotomy cut is an Evans osteotomy.

12. The osteotomy implant of claim 1, wherein the osteotomy implant is a prefabricated medical grade thermoplastic and/or polymer osteotomy wedge adapted to be implanted during surgical foot and/or ankle Cotton and/or Evans osteotomy bone corrections to stabilize and maintain flatfoot correction deformity procedures.

13. A method comprising:
making an osteotomy cut of a bone of a patient to achieve a correction;
selecting a trial wedge based on a characteristic of the cut, the patient, and/or a desired correction;
inserting the trial wedge into the cut;
determining whether an actual correction achieved with the trial wedge is acceptable;
selecting, when the actual correction achieved with the trial wedge is acceptable, a prefabricated osteotomy wedge implant based on a characteristic of the accepted trial wedge;
inserting the selected prefabricated osteotomy wedge implant into the cut to maintain the achieved bone correction;
the selected prefabricated osteotomy wedge implant comprising:
a first end;
a second end opposite the first end;
an opening at the second end, the opening extending toward the first end in plan view;
the selected prefabricated osteotomy wedge implant being composed of one or more medical-grade materials, the one or more medical-grade materials being radiolucent and/or osteoconductive; and
the selected prefabricated osteotomy wedge implant being adapted to maintain the bone correction achieved by the osteotomy cut.

14. The method of claim 13,
wherein the one or more medical-grade materials are osteoconductive; and
wherein the osteotomy wedge implant comprises a surface adapted to promote bone growth when the osteotomy wedge implant is inserted to maintain the bone correction achieved by the osteotomy cut.

15. The method of claim 14, wherein the opening has a first width in a plan view at the second end and a second width in the plan view at a middle portion of the osteotomy implant, the second width being wider than the first width.

16. The method of claim 15, wherein a portion of the opening is semi-circular in the plan view at the middle portion of the osteotomy implant, the second width being a diameter of the semicircular portion of the opening.

17. The method of claim 16,
wherein the selected prefabricated osteotomy wedge implant further comprising a surface that is semicircular in the plan view, the semicircular surface forming at least a portion of the semicircular portion of the opening, and
wherein the selected prefabricated osteotomy wedge implant further comprises a member coupled to the semicircular surface and extending across the semi-circular opening in the plan view.

18. The method of claim 13, wherein the one or more medical-grade materials comprises one or more of:
polyetheretherketone (PEEK), polyetherketoneketone (PEKK), and/or carbon fiber PEKK.

19. The method of claim 13, wherein the making an osteotomy cut of a bone to achieve a correction comprises making a Cotton osteotomy or an Evans osteotomy.

20. An osteotomy wedge kit for surgical foot and/or ankle osteotomy bone corrections, the osteotomy wedge kit comprising:
a plurality of osteotomy implants of two or more types/sizes;
a plurality of trial wedges, each of the trial wedges being configured to represent a respective one of the types/sizes of the implants; and
each osteotomy implant comprising:
a first end;
a second end opposite the first end;
an opening at the second end, the opening extending toward the first end in a plan view;
the osteotomy implant being composed of one or more medical-grade materials, the one or more medical-grade materials being radiolucent and/or osteoconductive; and
the osteotomy implant being adapted to hold a bone correction achieved by an osteotomy cut.
